(19) United States
(12) Patent Application Publication
(10) Pub. No.: US 2011/0318805 A1
(43) Pub. Date: Dec. 29, 2011

(54) Microbial biotransformation of spent vanilla beans

(76) Inventor: Ivica Labuda, Norwood, NJ (US)

(21) Appl. No.: 12/807,319

(22) Filed: Sep. 3, 2010

Related U.S. Application Data
(63) Continuation-in-part of application No. 11/440,769, filed on May 25, 2006, now Pat. No. 7,803,412.

(51) Int. Cl. C12P 1/04; C12P 1/02

(52) U.S. Cl. 435/170; 435/171

(57) ABSTRACT

A process for producing flavor constituents from spent vanilla materials, comprising treating the materials with microorganisms such as bacteria, fungi and yeast, that produce enzymes having the ability to degrade the plant cells systems of the materials, and recovering the flavor constituents so produced.

This application is a continuation-in-part of application Ser. No. 11/440,769, filed May 25, 2006 in the name of the present applicant, Ivica Labuda, which application is incorporated herein by reference in its entirety.

### FIELD OF THE INVENTION

The present invention relates generally to treatment of spent products normally discarded or used for a low-cost purpose in order to rejuvenate the products and obtain high-cost materials therefrom. More specifically it relates a microbial process for the production of natural flavor and modifier extracts obtained by the treatment of spent vanilla pods and beans normally discarded or used for low-cost animal feed.

### BACKGROUND OF THE INVENTION

Plants represent a rich pool of bioactive compounds. Vanilla is one of those plants; it is used for the production of natural vanilla flavor. Besides its obvious flavoring capacity, it can also be used as an anti-oxidant and microbial agent. Vanilla extract is obtained from cured vanilla pods by ethanol extraction. Vanilla extract contains about 60 to 80 different flavor components, of which vanillin is the major flavor component. In the past several decades a number of patents, e.g., U.S. Pat. Nos. 4,874,701, 4,981,7952, 195,871, 5,017,388, 5,262,315 and 5,128,253, and Japanese Patent Nos. 2,195,871 and 5,227,980, relate to the formation of vanilla flavors. These patents suggest various approaches using renewable, cheap and natural raw material sources such as ferulic acid, vanillic acid, vanillyl alcohol, eugenol, etc., employing either microorganisms, tissue cultures or enzymes, for vanillin formation.

Enzymes are widely used as a class of biocatalytic reagents in a wide variety of reactions including, e.g., oxidations, reductions, hydrolyses, and carbon-carbon bond ligations. Based on the nature of the solvent used, the enzymes can perform either a hydrolytic or a synthetic catalysis. Biocatalysts are valued for their intrinsic abilities to bind organic substrates and to catalyze specific and selective reactions under the mildest of reaction conditions. These selectivities and specificities are realized because of interactions occurring between the enzyme active site and the substrate molecule. Biocatalytic reactions are particularly useful when a traditional chemical catalysis is difficult and expensive to complete. Enzymes differ in their specificity towards substrates; some are highly selective while others accept a wide variety of substrates. In furtherance of that knowledge, I have treated spent vanilla materials with certain enzymes to obtain vanilla flavor constituents from materials that would otherwise have little if any commercial value.

Selective reduction of aromatic acids to their corresponding aldehydes by Actinomycetes sp. was reported by Jezo and Zemek (1986). Enzymes such as aryl-aldehyde oxidoreductase (carboxylic acid reductase EC 1.2.1.30) catalyze the formation of vanillin from vanillic acid (Rosazza et al., 1998, U.S. Pat. No. 5,795,759). Cellulases (endoglucanase, cellobiohydrolase, cellobiase, beta-glucosidase, etc.) on the other hand hydrolyze the O-covalent bond between glucose units in cellulose chains.

The development of the vanilla flavor during the curing treatment is partly due to the hydrolysis of a glucosylated precursor. An example of such glycoside is vanillin glucoside, which occurs in green vanilla pods (Arana. F. E., 1943, Food Research, vol. 8, pages 343-351). Precise biochemical pathways leading to all flavor components of vanilla are not known. Many flavor compounds are bound in plants and microorganisms, simply to protect the cells against the toxicity of these compounds. An enzyme breaking the glycosidic bond releases the flavor compound from its bond. An example of this phenomenon is an enzyme β-glucosidase, which hydrolyses the covalent bond of glucose from a β-aglycone. Two patents (U.S. Pat. No. 5,705,205 and FR-A-2,634,979) focused on the green vanilla beans and their curing process, during which the flavor develops. Flavor enhancement and efficiency of the curing process was central to these two patents.

At the industrial level, vanilla extract is obtained from vanilla pods that have been successfully cured. U.S. Pat. No. 5,705,205 suggests usage of β-glucosidase to shorten the curing process of green vanilla beans. Another patent FR-A-2,634,979 (1990) describes a process during which the green pods are frozen at a temperature of between −5 and −30 degrees C. and then reheated before extracting the flavor constituents therefrom. This process makes it possible to shorten the curing time and avoids losses of the flavor compounds which can occur during the curing process due to microbial or enzymatic decomposition. The typical curing process does not allow for the addition of external enzymes, only enzymes naturally present in the vanilla beans are employed in the curing process. Once the vanilla beans are cured the flavor compounds are extracted with ethanol. The remaining vanilla beans after the extraction are called spent or exhausted vanilla beans. These beans are typically used for animal feed and in very small quantities as food additives to give a visual impression of vanilla flavor, e.g., in vanilla ice cream. It is generally believed that most of the flavor is extracted during the extraction process and that these spent vanilla beans contain very minute amounts of flavor compounds.

Based on the forgoing background, it is an important object of the present invention to provide a process during which additional flavor compounds and food modifiers are released or transformed from complexes in spent vanilla beans and pods, thereby obtaining additional vanilla flavors and food modifiers from the spent materials.

However, while the object of the present invention is substantially the same as that of my prior, co-pending application, I have now discovered that, rather than employing enzymes to further break down the spent vanilla materials, those spent materials can be treated with enzymes indirectly by treating them with microorganisms that will themselves produce the enzymatic action that results in the production of flavor constituents. Microorganisms such as bacteria and yeast are capable of producing enzymes such as hydrolases, oxidoreductases and transferases. So, when treated with such microorganisms, stable substrates such as vanilla materials are metabolized or modified. Indeed, during this biotransformation process in which a substrate is treated with certain microorganisms, more than a single enzyme may be produced and the result is the achievement of a more varied and advantageous flavor than when a single enzyme is utilized.

### SUMMARY OF THE INVENTION

My invention is directed to a process for preparing natural flavor compounds from spent vanilla beans or pods which, in a complete, commercial process, comprises the following steps. First the spent vanilla pods and/or beans are ground or otherwise subdivided. Then they are hydrated to form a ground, hydrated product having a liquid phase and a solid phase. The next process step is treating the ground, hydrated product with microorganisms having the capacity to produce at least one enzyme capable of degrading the plant cells or transforming the flavor precursors. The microorganism is selected from the group consisting of bacteria, fungi and yeast and mixtures thereof. The enzyme produced by the microorganism is selected from the group consisting of an enzyme having pectinase activity, an enzyme having cellulase activity, an enzyme having hemicellulase activity, an enzyme having oxidoreductase activity, an enzyme having lipase activity, an enzyme having protease activity, and mixtures thereof. The subdivided, hydrated product containing the microbial system is incubated at general conditions of temperature that will vary, but will generally be that sufficient to promote microbial growth. Thereafter, the resultant mixture is separated and portions containing flavor constituents retained for further use.

### DETAILED DESCRIPTION OF PREFERRED EMBODIMENTS

The present invention is directed toward a process utilizing microorganisms such as bacteria, fungi and yeast to degrade and modify spent vanilla beans, pods and like materials. Typically the microorganisms such as bacteria, fungi or yeast generate one or more enzymatic systems chosen from hydrolases (E.C.3.), pectinases (polygalacturonase; EC 3.2.1.15), cellulases (1,4-[1,3;1,4]-β-D-glucan-4-glucano-hydrolase; EC 3.2.1.4), β-glucosidases, proteases (EC 3.4.23), hemicellulases (glycan hydrolase E.C. 3.2.1.), xylanase (1,4-β-D-xylanxylanohydrolase; EC 3.2.1.8), lipases (triacylglycerol lipase EC 3.1.1.3), esterases (E.C.3.1.), proteases (E.C.3.4.), oxidoreductases (EC 1.1.-EC 1.11), and transferases (E.C.2.). These enzymes may be used alone or in a mixture with β-glucosidase. These enzymes are well known to persons skilled in the art and many systems based thereon are commercially available. The broader implication of this discovery includes the economical production of products and intermediary by-products.

It is understood that the invention covers microbes capable of producing all the enzymatic systems, which make it possible to degrade and transform the precursors potentially contained in the spent (exhausted) vanilla pods and beans. Persons skilled in the art will be able, using simple procedures such as those defined in the example which follows, to choose the microbial systems that are appropriate.

### Example

Cells of the fungus Aspergillis niger were inoculated into 200 ml. of mineral medium consisting of (NH₄)₂SO₄ at 5 g/l, KH₂PO₄ at 0.2 g/l, CaCl₂.2H₂O at 0.0132 g/l, MgSO₄.7H₂O at 0.5 g/l, yeast extract at 0.5 g/l and glucose at 20 g/l. The cells were grown on a rotary shaker for 40 hours at 30° C. The mycelium produced was collected by filtration and added to 180 ml of water containing 20 g of spent vanilla beans and 0.5 g of glucose. A slurry consisting of two phases, one solid and one liquid, was stirred. The incubation with the cells was typically carried out without pH adjustment as the natural pH was around 5.0. The pH was measured after addition of the cells and at the end of the biotransformation and recorded. The slurry was agitated while incubated at 30° C. for 14 hours. At the end of the reaction the mixture was mixed with 96% (v/w) ethyl alcohol to obtain a 50% (v/w) aqueous alcohol solution. This solution was then stirred for three hours at room temperature and filtered through a Whatman paper filter. The solute contained notes reminiscent of vanilla extract.

Any microorganisms capable of producing any of the enzymes mentioned hereinbefore are appropriate for use in the present invention to generate vanilla flavor constituents from spent vanilla materials. Microorganisms that have been determined to be appropriate are, without limitation, Bacillus sp., Escherichia coli, Streptomyces sp., Thermomyces sp., Aspergilus sp., Trichoderma sp., Mucor sp., Chaetomium sp., Polyporus sp., Pychnoporus sp., Trametes sp., Myceliophthera sp., Rhizoniucor sp., and Candida sp. It will thus be apparent that the microorganisms can be selected from bacteria, fungi and yeast.

When microbial cells are utilized in accordance with my invention, it has been found that the best results have been obtained when the microbial cells are in stationary cells. The microbial cells are maintained during the biotransformation process in a liquid medium containing substances necessary to support the viability of the microorganisms, such media being, for example, nutrient substances, minerals, cofactors, and the like. The general requirements for maintaining the viability of cells is well established an easily determined by a microbiologist for each type of microorganism utilized. Preferably the medium for the microorganism includes YP medium containing assimilable sugars such as sucrose and glucose.

Temperatures from about 10° C. to about 40° C. for a period of from about 2 hours to about 48 hours, have been usually been found to be sufficient to enable the microorganism to enzymatically transform the flavor precursors and allow the release of natural flavor compounds in the hydrated product. The mixture of solid and liquid phases can be mixed with ethyl alcohol at a concentration of from about 1% to 50% product to enhance the release of natural flavor compounds. Thereafter the liquid phase is separated from the solid phase of the mixture, and the liquid phase that contains the natural flavor extracts together with other soluble materials is recovered. Within these extracts and materials further precursors to other flavors can be present. Some of these flavor compounds can modify taste perception.

With respect to the enzymatic systems produced by the microorganisms, a further description of those enzymes and the optimum conditions for their maintenance is found is my application Ser. No. 11/440,769, of which the present application is a continuation-in-part and which parent is incorporated herein in its entirety.

It will be apparent to those of skill in this art that the foregoing disclosure is only illustrative of my invention and that modifications thereof and additions thereto will be apparent to those of skill in this art. As to all such modifications and additions, it is desired that they be included within the purview of my invention, which is to be limited only by the scope, including equivalents, of the following, appended claims.

I claim:

1. A process for recovering flavor constituents from spent vanilla materials, comprising forming a mixture of such spent materials, treating the mixture with microorganisms selected from the group consisting of bacteria, fungus and yeast to produce an enzyme having the ability to degrade the plant cell systems of said materials, continuing such treatment for period of time and at a pH and temperature such that said cell systems break down and release flavor constituents, and thereafter recovering said flavor constituents from said mixture.

2. A process as claimed in claim 1, in which said enzyme is selected from the group consisting of a hydrolase, oxidoreductase and transferase and mixtures thereof.

3. A process as claimed in claim 2, in which said enzyme is a hydrolase.

4. A process as claimed in claim 3, in which said enzyme is selected from the group consisting of hydrolases with glucosidase, cellulase, pectinase, hemicellulase, xylanase, protease and lipase activity.

5. A process as claimed in claim 3, in which said enzyme is β-glucosidase.

6. A process as claimed in claim 2, in which said enzyme is an oxidoreductase.

7. A process as claimed in claim 2, in which said enzyme is a transferase.

8. A process as claimed in claim 1, in which said vanilla materials are spent vanilla pods and beans.

9. A process as claimed in claim 8, in which said mixture of spent vanilla materials is treated with said microorganism for a period of about 1 to 120 hours at a temperature of about 10° to 100° C.

10. A process as claimed in claim 9, in which said mixture is treated with said microorganism at a pH between about 3 and 10.

11. A process as claimed in claim 10, in which said pH is about 5.

12. A process as claimed in claim 8, in which said spent vanilla materials are spent pods and beans ground to increase exposure to said enzyme.

13. A process for obtaining flavor constituents from spent vanilla materials, comprising grinding the vanilla materials to increase their exposure to enzymatic activity, forming an aqueous mixture of said ground materials, agitating the mixture to form a slurry consisting of liquid and solid phases, treating the slurry with a microorganism selected from the group consisting of bacteria, fungi and yeast and combinations thereof, said microorganism having the ability to produce an enzyme capable of degrading the plant cell systems of said materials, maintaining said treated slurry for period of time and at a pH and temperature such that said microorganism produces an enzyme in a quantity sufficient to break down said plant cell systems and release said flavor constituents, incubating said slurry for a period of time sufficient to substantially complete said reaction and release of said constituents, and thereafter recovering said flavor constituents from said mixture.

14. A process as claimed in claim 13, in which said enzyme is selected from the group consisting of β-glucosidase and mixtures of β-glucosidase and other enzymes,

15. A process as claimed in claim 13, in which said microorganism slurry includes glucose or sucrose.

16. A process as claimed in claim 15, in which said microorganism slurry includes hydrates of sulfates and chlorides.

17. A process as claimed in claim 13, in which said microorganism slurry is incubated at about 35° C. for about 20 hours.

18. A process as claimed in claim 13, in which after incubation said mixture is separated into liquid and solid phases and said flavor constituents are recovered from said liquid phase.

19. A process for recovering flavor constituents from vanilla beans and pods, comprising extracting vanilla flavors from said beans and pods by ethanol extraction to form spent vanilla materials as a byproduct, and thereafter slurrying said spent materials with a microorganism selected from the group consisting of bacteria, fungi and yeast to produce an enzymes having the ability to degrade the plant cell systems of said materials, incubating said slurry for period of time and at such a pH and temperature that said plant cell systems break down and release flavor constituents, and thereafter recovering said flavor constituents from said slurry.
